SEQUENCE LISTING (12 sequences)

SEQ ID NO: 1 - DNA, 4711 bp, Homo sapiens
agtgagctatggggacactactgcactgtagcctgggcaacagagcaagaccttgtctcaaaaatgtatatatattttgggctttttttcctaaaacgggaactacaacagcatatttgcgagctgatgagagtgacccagcagagagggaaatggatcagctctgttgaagatgcactggacaccagaacacgcccagcccctcaaccagtggccagagcagcacctggacgtctcctccaccaccccgtcgccggcccacaagttggagttgccccctgggggtcgccaacgctgccactacgcttgggcacacgacgacatctcagccctcactgcctccaacctcctaaagcgctatgcagagaagtactctggggtcttggattctccctacgagcgtccggccctgggcgggtacagcgacgcctccttcctcaacggcgccaaaggggatcccgagccctggccagggccggagccaccctaccccttggcctcactccacgaaggcctcccaggaaccaaatcgggcggtggcggcggttccggggccctggggggctccccagttttagccgggaacctccctgaacccctctacgccggcaatgcgtgcgggggcccatcggcggcgcccgagtacgcggccggctacggcggggggtacctggcgccgggttactgcgcgcagacgggcgccgcgctgcccccgccgcccccggccgcgctcctgcagcccccaccgcctccggggtacgggccctcagcgccgctgtacaactatcccgcagggggctacgcagcgcagcccggctatggcgcgctcccgccgcccccaggcccacccccggccccctacctgaccccgggcctgcccgcgcccacgcccctgcccgcgccggcaccgcccaccgcctatggcttccccacggccgcgccgggtgccgaatccgggctgtcgctgaagcgcaaggccgccgacgaggggcccgagggccgctaccgcaagtacgcgtacgagcccgccaaggcccccgtggctgacggagcctcctaccccgccgcggacaacggcgaatgtcggggcaacgggttccgggccaagccgccaggagccgcggaggaggcgtcgggcaagtacggtggcggcgtccccctcaaggtcctgggctcccccgtctacggcccgcaactggagccctttgaaaagttcccggagcgggccccggctcctcgtggggggttcgccgtgccgtcgggggagactcccaaaggcgtggaccctggggccctggagctggtgacgagcaagatggtggactgcgggcccccggtgcagtgggcggatgtggcgggccagggcgcgctcaaggcggcgctggaggaggagctggtgtggcccctgctcaggccgcccgcctacccgggcagcctgcgcccgccgcggaccgtcctgctctttgggccgcggggcgcgggcaaagcgctgctgggccgctgcctcgccacgcagctgggcgccacgctgttgcgcctgcgcggcgcgaccctggctgcgcccggcgccgccgagggcgcgcgcctcctccaggccgccttcgcggccgcgcgctgccgcccaccctccgtactcctcatcagcgagctagaggcgctgctccccgcccgggacgacggcgcggcggcagggggcgcgctgcaggtgccgctcctggcctgcctggacgggggctgcggcgcgggggctgacggcgtgctggttgtgggcaccacctcgcggcccgcggctctggacgaggcgacccgccggcgcttctctctccgcttctacgtggcgctgcccgacagcccggcccgcgggcagatcctgcagcgggcgctggcccagcagggctgcgcgctcagtgagcgggaactggcggcgctggtgcagggcacgcagggcttctctgggggcgagctggggcagctgtgccagcaggcggcggccggggcgggcctcccggggctgcagcgccccctctcctacaaggacctggaggcggcgctggccaaggtgggccctagggcctctgccaaggaactggactcgttcgtggagtgggacaaaatgtacggctccggacactgacggcgcgcgggggaggccgcgggagccgcagtccctccgtccccgccgcctccgcgtgggagggatgtcactgactaaacccggctggcaggggctggagtggtgaatgtgggatcggggacaggaggggtctgccggtggatattttttttttcgtgggaaggaaaatgcttctgccaggcagatgccatatgcgccgtgtactcaggtttttcctatttattgtggactggaagctcgccatctccgcccggcagaccgggcagatccggcatgggctggcacccggggccttaagaactcctgctctcttgccacaacgcttttgtctcctcgctatctgaatggcaccctccttctccctcactctctccatcccattctctgcattctcttggttttctctcccttttgctttgtcgctgacacccctgcccaccccatgctggccctgtttctctcctgcccctccctccccagctctccatccctcaccctctgtgcttctgtctccatccctggctctccagcgtccctggccttttggtccctgagctttaatgcctttccctgccttctgttcttatttggactgcagtggccctttgcaggagctctggaggcccaggggctgaggaggagggttacccctctacccatctgaaacctagggtctagggggatcaaggaaaaaaagtccccaaagaaggggaattttttgtttgtttttgaggggagatcccagaaatgtagcttgtttcatattttagtcttcttatttttgtaaaatgtgtagaatttgctgtttttctttttcttttgacaactcaggaagaaactgacctcagaaagaatgttagactttggctgctctcctgtgtgcccctcacacctgccccctcccccccactccatccaggggaccaaattctcccagacactcaaaaaatgagacttacggggaaggggagaggaagacccagaggcctcagtgaaaccccagctattcctggtcagaagcagaatgtattcctaagggcttcctccccagggccgaggcctaggcatgaatgtggggagtgggctgtggggtttgagagaagggaggccttattcctctcctgctgctccccaccccctgccccacccaacccctccgctgagtgttttctgtgaagggctatccagagttaggatgcccttgcccaattccttcctgagacccagaaggtagggtgggagggcccaaatgggaaggtgacctaagcagaaagtctccagaaaggtcatgtcccctggccctgccttggcagaggtccccagtgacttatgctaggaggattccatctgggtagacagtctggccacaaaatcagctactggacctcagccatctctgctggaggctctgaggaggagtgagcatccctcacttgtgggggctctgtgaggaaatgtgccttccccattcccccggagtcctaggtctggagctccagggctgggagagggtgagggagatgggcaggggtgttttctctgaccttgggggcttagtctcagtcctgcctgaactttccactaggcttggaacccttccaagaaccatatttctctccttcccaccaattttcccttgatgaggctttagcagtttgctcccaccacccccagcccatttcacaactctgatcttagtccaaagcaggggacacgcccccccaccaccactttttctctctcccatctcagcctcctgtgcagttccttgcctgcccgtgcatttcctagagtctactgcctcccccctggctgggagggtgtctgggggggatctttcaggggccctggcacccagggcctgtgctggcctaggagtgctgaccagaaggctgctctgttcccccccacccccgttgctttctggccccctctttggagccagccacccacagggctttggtgcctcagaagcagtgggctgccgggtcacagccgcaggctgcaaaagaccctcggagggagcatggagtgaggggttctctctcaggtgtgtatgtattggggggtgggggtgggtggagggtgtcagggaagttggggtgggatcccagccttcccttcaagaggcagggagctctgggaggtggagtccccaccgctttctctactaggctcctcctgttccccaggcttggggagctttgcacaaggagactgcccccagcctagtggcacctacctcatgggctctggggcaggtaggggaagggccagtccagctctggtaatgctggggggaggcataccaaagaatccaggggcagggagtggggagggtgacttccgagctggcctctccccttcctctacccagactggggctgggatcctctcctcccgctgtaaccatttctacctcattttgctgcgtgttgtacatggacgtatttatctcctgtctgacgatgctctgcagttgtggtctgtctacctcagaagagactgtattttaaaagaaagtattacacagtattaaagcgatgacatgtggtttgcaaaaaaaaaaaaaaaaaa

SEQ ID NO: 2 - protein, 653 aa, Homo sapiens
MHWTPEHAQPLNQWPEQHLDVSSTTPSPAHKLELPPGGRQRCHYAWAHDDISALTASNLLKRYAEKYSGVLDSPYERPALGGYSDASFLNGAKGDPEPWPGPEPPYPLASLHEGLPGTKSGGGGGSGALGGSPVLAGNLPEPLYAGNACGGPSAAPEYAAGYGGGYLAPGYCAQTGAALPPPPPAALLQPPPPPGYGPSAPLYNYPAGGYAAQPGYGALPPPPGPPPAPYLTPGLPAPTPLPAPAPPTAYGFPTAAPGAESGLSLKRKAADEGPEGRYRKYAYEPAKAPVADGASYPAADNGECRGNGFRAKPPGAAEEASGKYGGGVPLKVLGSPVYGPQLEPFEKFPERAPAPRGGFAVPSGETPKGVDPGALELVTSKMVDCGPPVQWADVAGQGALKAALEEELVWPLLRPPAYPGSLRPPRTVLLFGPRGAGKALLGRCLATQLGATLLRLRGATLAAPGAAEGARLLQAAFAAARCRPPSVLLISELEALLPARDDGAAAGGALQVPLLACLDGGCGAGADGVLVVGTTSRPAALDEATRRRFSLRFYVALPDSPARGQILQRALAQQGCALSERELAALVQGTQGFSGGELGQLCQQAAAGAGLPGLQRPLSYKDLEAALAKVGPRASAKELDSFVEWDKMYGSGH

SEQ ID NO: 3 - RNA, 21 nt, Artificial
  Synthetic siRNA
uuacacaguauuaaagcgauu

SEQ ID NO: 4 - RNA, 21 nt, Artificial
  Synthetic siRNA
ucgcuuuaauacuguguaauu

SEQ ID NO: 5 - RNA, 21 nt, Artificial
  Synthetic siRNA
caucugaaaccuagggucuuu

SEQ ID NO: 6 - RNA, 21 nt, Artificial
  Synthetic
agacccuagguuucagauguu

SEQ ID NO: 7 - RNA, 21 nt, Artificial
  Synthetic
gugacuuaugcuaggaggauu

SEQ ID NO: 8 - RNA, 21 nt, Artificial
  Synthetic
uccuccuagcauaagucacuu

SEQ ID NO: 9 - RNA, 21 nt, Artificial
  Synthetic
ggucagaagcagaauguauuu

SEQ ID NO: 10 - DNA, 21 bp, Artificial
  Synthetic
auacauucugcuucugaccuu

SEQ ID NO: 11 - DNA, 23 bp, Artificial
  Synthetic
cagcucgagcccuuugacadtdt

SEQ ID NO: 12 - DNA, 23 bp, Artificial
  Synthetic
ugucaaagggcucgagcugdtdt

(19) United States
(12) Patent Application Publication
(10) Pub. No.: US 2021/0353824 A1
(43) Pub. Date: Nov. 18, 2021

(54) FIDGETIN-LIKE 2 SIRNA-ENHANCED POLOXAMER-BASED HYDROGEL FOR WOUND HEALING

(71) Applicants: ALBERT EINSTEIN COLLEGE OF MEDICINE, Bronx, NY (US); MICROCURES, INC., Santa Cruz, CA (US)

(72) Inventors: David James SHARP, Scarsdale, NY (US); Brian O'ROURKE, Boston, MA (US); Adam Hildyard KRAMER, Queens, NY (US)

(73) Assignees: ALBERT EINSTEIN COLLEGE OF MEDICINE, Bronx, NY (US); MICROCURE, INC., Santa Cruz, CA (US)

(21) Appl. No.: 17/287,910

(22) PCT Filed: Oct. 23, 2019
(86) PCT No.: PCT/US2019/057611

§ 371 Date: Apr. 22, 2021

Related U.S. Application Data
(60) Provisional application No. 62/749,325, filed on Oct. 23, 2018.

(51) Int. Cl. A61L 26/00; A61K 8/02; A61K 8/60; A61K 8/90; A61K 8/65

(52) U.S. Cl. CPC A61L 26/0033; A61K 8/0241; A61K 8/606; A61K 8/90; A61L 2300/412; A61L 26/0066; A61L 2300/258; A61L 2300/406; A61K 8/65

(57) ABSTRACT

Methods and compositions for reducing a ratio of collagen III to collagen I in wounded skin, treating wounds and accelerating healing is provided with a surfactant polymer dressing comprising FL2 siRNA, collagen and a poloxamer.

### CROSS-REFERENCE TO RELATED APPLICATION

This application claims priority to U.S. application Ser. No. 62/749,325, filed Oct. 23, 2018, which is incorporated herein by reference in its entirety.

### BACKGROUND OF THE INVENTION

Wound healing is a complex physiological process which, when not effectively completed, can lead to serious complications such as chronic wounds, infections, and ultimately amputations. Current approaches in acute wound healing are generally limited to passive dressings, skin-like matrices, and growth factor-based approaches that address only the initial, inflammatory phase of the healing process. Improved wound healing methods and compositions are needed.

The present invention addresses this need and identifies a novel target in promoting wound healing and provides therapies and assays based thereon.

### SUMMARY OF THE INVENTION

A cosmetic composition or pharmaceutical composition is provided comprising (i) a siRNA directed to fidgetin-like 2 (FL2) associated with, or adsorbed to, a collagen particle in (ii) surfactant polymer dressing.

A method is provided for reducing a ratio of collagen III to collagen I in wounded skin during healing of a mammalian skin wound comprising a burn, the method comprising applying an amount of cosmetic composition or pharmaceutical composition comprising (i) a siRNA directed to fidgetin-like 2 (FL2) associated with, or adsorbed to, a collagen particle in (ii) surfactant polymer dressing, effective to reducing a ratio of collagen III to collagen I during healing of the burn wound.

A method is provided for treating a mammalian skin wound comprising applying an amount of the cosmetic composition or pharmaceutical composition (i) a siRNA directed to fidgetin-like 2 (FL2) associated with, or adsorbed to, a collagen particle in (ii) surfactant polymer dressing, effective to treat the mammalian skin wound.

A method is provided for accelerating healing of a mammalian skin wound comprising applying an amount of the cosmetic composition or pharmaceutical composition (i) a siRNA directed to fidgetin-like 2 (FL2) associated with, or adsorbed to, a collagen particle in (ii) surfactant polymer dressing effective to accelerate healing of the mammalian skin wound.

A method is provided for decreasing inflammation and/or increasing in re-vascularization at a skin wound site, comprising applying an amount of the cosmetic composition or pharmaceutical composition (i) a siRNA directed to fidgetin-like 2 (FL2) associated with, or adsorbed to, a collagen particle in (ii) surfactant polymer dressing, effective to decrease inflammation and/or increase re-vascularization at the skin wound site.

A wound dressing structure is provided comprising a portion having thereon a composition comprising (i) a siRNA directed to fidgetin-like 2 (FL2) encapsulated by collagen microparticles in (ii) surfactant polymer dressing.

### BRIEF DESCRIPTION OF THE DRAWINGS

FIG. 1A-1B. FL2 levels are reduced in injured skin treated with SPD-FL2-siRNA. FIG. 1A) Schematic of microparticle siRNA incorporation into SPD. FIG. 1B) Mice were burned then treated at day 0 and day 2 with the designated agent. Burns were harvested on day 3 and RNA isolated for subsequent qPCR analysis. SPD-FL2-siRNA treatment significantly reduces FL2 mRNA relative to SPD-Control-siRNA treatment. N≥8; nonparametric Mann-Whitney test; p=0.026; Error bars=S.E.M.

FIGS. 2A-2B-2C. SPD-FL2-siRNA Treatment Stimulates Healing in a Mouse Full-Thickness Excision Model. FIG. 2A) Representative time course of healing following application of the surfactant polymer treatment in BALB/c mice. FIG. 2B, FIG. 2C) Graphs quantifying remaining wound area over time and at day 4. Red bars are SPD treatment, blue bars SPD-Control-siRNA, green bars SPD-FL2-siRNA. N=22 wounds/group; Student's t-test; ***p≤0.0008; Error bars=S.E.M. Significance confirmed at individual timepoints and with a two way ANOVA.

FIG. 3A-3B. SPD-FL2-siRNA Treatment Reduces Wound Size in Mouse Full-Thickness Excision Model. FIG. 3A) Representative H&E staining of bisected wounds taken 8 days after wounding. Epithelial thickening is present in the epidermis of the control groups while FL2-siRNA treated animals show re-epithelialization indicative of a further progressed, well healing wound. Arrows designate observable wound areas. Inset image highlights keratin 14 positive hair follicles within the wound zone. FIG. 3B) Graph quantifying wound length at day 8. N≥4; Student's t-test; p *=0.0252; **=0.0079; Error bars=S.E.M.

FIG. 4A-4B-4C-4D. SPD-FL2-siRNA Treatment Stimulates Healing in Mouse Full-Thickness Burn Model. FIG. 4A) Representative H&E staining of bisected wounds taken 14 days post burning. FL2-siRNA treated animals show re-epithelialization indicative of a further progressed, well healing burn. Arrows designate observable wound edges. FIG. 4B) Graph quantifying wound area over time relative to day 0. N≥14; Significance confirmed at individual timepoints and with a two-way ANOVA. FIG. 4C, FIG. 4D) Graphs quantifying wound area at day 4 and epidermal thickness at day 14. Red bars are SPD treatment, blue bars SPDControl-siRNA, green bars SPD-FL2-siRNA. N≥8; Student's t-test; *p≤0.05; **p≤0.005; ***p≤0.0005; ****p≤0.00005; Error bars=S.E.M.

FIG. 5A-5B-5C-5D. FL2 knockdown Enhances Revascularization and Reduces Inflammation Without Affecting Cell Proliferation FIG. 5A) Immunohistochemical staining of skin 14 days post burn and treatment using either PCNA, PECAM1, or CD45 antibodies, respectively. Central regions of the burns were imaged at 20× or 40× magnification and the number of staining events quantified. FIG. 5B) PCNA staining showed no significant differences between treatment groups. N≥4 wounds per condition, ≥3 fields per wound; Student's t-test; n.s.=not significant. FIG. 5C) Significant differences were observed between both controls and SPD-FL2-siRNA treatment groups following PECAM1 staining. N≥5; Student's t-test; **p=0.0092). FIG. 5D) Significant differences were observed between control and SPD-FL2-siRNA treatment groups following CD45 staining. N≥10; Student's t-test; p=**0.0027; ***0.0003; Error bars=S.E.M.

FIG. 6A-6B. SPD-FL2-siRNA Treatment Enhances Collagen Remodeling in Mouse Full-Thickness Burn Model. FIG. 6A) Representative Herovici staining of bisected wounds taken 14 days post burning. FL2-siRNA treated animals show improved collagen maturation with a reduced collagen III (blue) to collagen I (red) ratio. Insets show 40× magnified view of wound zone. FIG. 6B) Graph quantifying collagen ratio within the wound. N≥11; Student's t-test; ***p=0.001; Error bars=S.E.M.

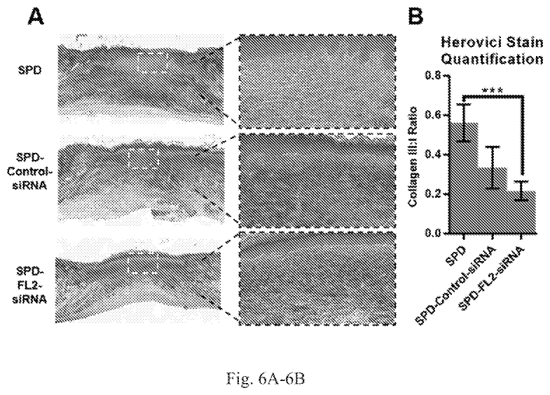

### DETAILED DESCRIPTION OF THE INVENTION

Slow healing and open wounds are a serious medical problem faced by patients worldwide. Without proper re-epithelialization, these wounds are at risk for infection, increasing the risk of amputation or mortality. Slow healing wounds also bring the risk of scarring and skin deformation. A therapeutic which can more quickly close wounds would directly address these issues.

As will be seen in the examples herein, treating wounds with a surfactant polymer dressing (SPD) provides significant hydration to the wound site and increases antimicrobial efficacy, improving healing outcomes. The data shown here demonstrate that incorporation of FL2 siRNA, into a surfactant polymer dressing further improves wound healing outcomes.

The clinical and histological characterization of treated wounds presented herein indicate that surfactant polymer dressing comprising FL2 siRNA (SPD-FL2-siRNA) stimulates more efficient re-epithelialization and progresses wounds into subsequent stages of healing (resolution of inflammation) and regeneration (collagen maturation, angiogenesis) more quickly than controls.

The extent of inflammatory response following injury is a first indicator of potential wound outcome. It is noteworthy that SPD-FL2-siRNA treated wounds show an expedited resolution of inflammation at earlier time points and reduced CD45+ staining at later points; there is now evidence that more effective re-epithelialization is correlated with shortened periods of inflammation and that reduced CD45+ levels 7 days post-injury is indicative of further progressed wounds. Next, as re-epithelialization continues, leading edge cells generate a “healing” scaffold into the wound zone. This structure creates a path for cells to efficiently migrate to the center of the wound and begin remodeling the ECM matrix. The findings here show that FL2 knockdown influences collagen organization within the wound zone, reducing the ratio of collagen III to collagen I in treated wounds. While increased deposition of collagen III is known to reduce the density of blood vessels within the wound collagen I is an optimal substrate for endothelial cells and this may explain the increase in angiogenesis seen in SPD-FL2-siRNA treated wounds.

Additional cell types, such as epidermal stem cells, also utilize this newly formed scaffold to migrate into the wound zone. We found that only FL2 siRNA treated wounds showed stem cell derived hair follicles within the wound zone, suggesting these wounds were further progressed in the regeneration process. Re-epithelialization also impacts local cellular signaling within the wound zone. Increasingly present keratinocytes release VEGF, recruiting endothelial cells into the wound zone, and may provide an alternative explanation for the increased angiogenesis seen in SPD-FL2-siRNA treated wounds.

A new SPD-siRNA platform that can be applied topically and used to treat excision wounds and burns is provided in one embodiment. Treatment with SPD-FL2-siRNA depletes FL2 levels, which expedites re-epithelialization and ultimately improves dermal structure compared to treatment with SPD alone. Due to its favorable chemical composition and ease of application, an SPD-FL2-siRNA product could improve the lives of patients suffering from a variety of dermal injuries.

The invention is in some embodiments directed to a cosmetic composition or pharmaceutical composition comprising (i) a siRNA directed to fidgetin-like 2 (FL2) associated with, or adsorbed to, a collagen particle in (ii) surfactant polymer dressing.

In some embodiments, the cosmetic composition comprises a cosmetic product.

In some embodiments, the pharmaceutical composition comprises a pharmaceutical carrier.

In some embodiments, the collagen particle comprises reversible micelles. In some embodiments the collagen particle comprises AOT.

In some embodiments, the surfactant polymer dressing comprises a poloxamer. In some embodiments, the poloxamer comprises hydrophilic polyethylene oxide and hydrophobic polypropylene oxide. In an embodiment, the poloxamer is a synthetic tri-block copolymers composed of a central hydrophobic chain of polyoxypropylene flanked by two hydrophilic chains of polyoxyethylene with a weight ratio of 4:2:4. In an embodiment, the poloxamer is poloxamer 188. In an embodiment, the poloxamer has an average molecular weight of 8400 Daltons. In some embodiments, the surfactant polymer dressing comprises PluroGel®.

In some embodiments, the poloxamer is present at about 10% in an aqueous medium. In some embodiments, the aqueous medium is water.

In some embodiments, the surfactant polymer dressing further comprises 4-(1,1,3,3-tetramethylbutyl)phenyl-polyethylene glycol. In some embodiments, the 4-(1,1,3,3-tetramethylbutyl)phenyl-polyethylene glycol has a molecular weight of about 647 wherein 10 repeating polyethylene oxide groups are present. In some embodiments, an average of 9.5 polyethylene oxide groups are present. In some embodiments, the surfactant polymer dressing further comprises Triton-100.

In some embodiments, the surfactant polymer dressing is a poloxamer, a meroxapol, or a poloxamine.

Non-limiting examples of poloxamers include poloxamer-101, -105, -105 benzoate, -108, -122, -123,-124, -181, -182, -182 dibenzoate, -183, -184, -185, -212, -215, -217, -231, -234, -235, -237, -238, -282, -284, -288, -331, -333, -334, -335, -338,-401,-402, -403, and -407. In another embodiment, the poloxamer is poloxamer-407.

Exemplary meroxapols include, but are not limited to, meroxapol 105, 108, 171, 172, 174, 178, 251, 252, 254, 258, 311, 312, and 314.

Exemplary poloxamines include, but are not limited to, poloxamine 304, 504, 701, 702, 704, 707, 901, 904, 908, 1101, 1102, 1104, 1301, 1302, 1304, 1307, 1501, 1502, 1504, and 1508.

In some embodiments, the collagen microparticle comprises collagen I in a sodium bis(2-ethylhexyl) sulfosuccinate (AOT; docusate sodium) membrane. In some embodiments, a collagen solution in acetic acid is dissolved in a solution of AOT in hexane. In some embodiments, the resulting microemulsion is evaporated, suspended in water and lyophilized.

In some embodiments the microparticles comprise micelles. In some embodiments, the microparticles comprise a non-ionic compound such as but not limited to gum arabic, alginic acid, cetostearyl alcohol, cetyl alcohol, a glucose fatty acid ester, glyceryl monooleate, glyceryl monostearate, hydroxypropyl cellulose, a medium chain triglyceride, low molecular weight methylcellulose, a polyoxyethylene alkyl ether, a polyoxyethylene castor oil derivative, a polyoxyethylene fatty acid ester, a polyoxyethylene stearate, polyvinyl alcohol, a sorbitan fatty acid ester, or a sucrose fatty acid ester; or mixtures thereof. In some embodiments the microparticle membrane comprises a cationic compound, such as but not limited to cetrimide, monoethanolamine or triethanolamine; or mixtures thereof. In some embodiments the microparticles comprise an anionic compound such as but not limited to a cholic acid derivative, carbomer, oleic acid, propylene glycol alginate, sodium dodecyl sulfate, stearic acid, white wax or yellow wax; or mixtures thereof. In some embodiments, the microparticles comprise a mixture of compatible compounds from any of the foregoing selections.

In some embodiments the collagen microparticles are loaded with FL2 siRNA. In some embodiments the lyophilized microparticle powder is treated with siRNA solution in nuclease free water. In some embodiments the siRNA-loaded particles are then lyophilized.

In some embodiments, the lyophilized siRNA-loaded collagen particles are mixed with the surfactant polymer dressing. In one embodiment, the lyophilized particles are mixed to the desired concentration with combination of an equal amount of water and surfactant polymer dressing.

In some embodiments, the siRNA-collagen microparticle-SPD composition may comprise one or more additional components. In some embodiments, additional components include one or more of the following: antibiotics, antiseptics, vitamins, anesthetics, antihistamines, anti-inflammatory agents, moisturizers, penetration-enhancing agents, sunscreens, and/or anti-irritants. In an embodiment, the compositions do not comprise further active ingredients suitable for accelerating recovery from a skin wound, for example one or more antibiotics, antiseptics, vitamins, anesthetics, antihistamines, anti-inflammatory agents, moisturizers, penetration-enhancing agents, sunscreens and/or anti-irritants.

In some embodiments, the collagen particles are microparticles. In some embodiments, the microparticles have sizes from about 1 μm to about 1000 μm. In some embodiments, the microparticles have sizes from about 1 μm to about 100 μm. In some embodiments, the microparticles have sizes from about 1 μm to about 50 μm. In some embodiments, the microparticles have sizes from about 1 μm to about 40 μm. In some embodiments, the microparticles have sizes from about 3 μm to about 100 μm. In some embodiments, the microparticles have sizes from about 3 μm to about 40 μm. In some embodiments, the microparticles have sizes from about 1 μm to about 5 μm. In some embodiments, the microparticles have sizes from about 3 μm to about 5 μm. In some of these embodiments, the size is the average size or average diameter of the microparticles.

In some embodiments the collagen particles are a microemulsion. In some embodiments, the particles have sizes from about 1 nm to about 1000 nm. In some embodiments, the particles have sizes from about 1 nm to about 500 nm. In some embodiments, the particles have sizes from about 1 nm to about 300 μm. In some embodiments, the particles have sizes from about 10 nm to about 500 nm. In some embodiments, the particles have sizes from about 10 nm to about 300 nm. In some embodiments, the particles have sizes from about 10 nm to about 100 nm. In some embodiments, the particles have sizes from about 10 nm to about 30 μm. In some of these embodiments, the size is the average size or average diameter of the particles.

In some embodiments, the siRNA/collagen/SPD is applied to the site of an injury. In some embodiments, the injury is a wound or burn (hereinafter referred to as a wound to encompass all embodiments thereof). In some embodiments the wound is an abrasion, a cut, an excisional wound or multiple lacerations.

The FL2 siRNA-collagen microparticle-surfactant polymer dressing, referred to herein as a pharmaceutical composition, may be used in some embodiments for application to a wound or burn to accelerate healing. In some embodiments, the pharmaceutical composition may be applied to the wound once or more than once, daily, twice a day, three times a day, four times a day, or more times a day. In some embodiments the pharmaceutical composition may be applied daily or more frequently during the day, for a duration of time until the wound is healed, or for a period between one day and when the wound is healed. In some embodiments, the pharmaceutical composition may be applied to the wound less frequently than once a day, wherein on the day of application (an application day), the frequency of applications during the day is any of the foregoing frequencies. In one embodiment, application days are every other day, or every third day, or every fourth day, or less frequently. In some embodiments the pharmaceutical composition may be applied on application days less often than daily and more frequently during the application day, for a duration of time until the wound is healed, or for a period between one day and when the wound is healed. The number for applications per day, the number of days when at least one application is applied, and the duration of treatment, may be governed by the rate of healing of the wound, the appearance of the wound, any irritation or other undesirable aspect of application of the pharmaceutical composition, or any other factor.

On days or periods during the day when the wound is not being treated with the pharmaceutical composition, a different pharmaceutical composition may be applied, such as a moistener, antibiotic ointment, anti-inflammatory ointment, or any other wound care product. A surfactant polymer dressing with or without collagen microparticles, either without siRNA, may be used between applications of the siRNA to maintain a consistent coverage of the wound.

In some embodiments, after application of the pharmaceutical composition, an occlusive or non-occlusive dressing may be placed over the treated wound to protect the site from inadvertent removal of the pharmaceutical composition, or to maintain the pharmaceutical composition in place. An adhesive bandage, taped on gauze, a hydrogel dressing, or any other type of wound dressing may be applied to maintain contact of the pharmaceutical composition with the wound and prevent removal, contamination, among other factors to protect the wound from direct external contact during the healing process.

In some embodiments, the pharmaceutical composition described herein is called a cosmetic composition. In some embodiments, it is called a pharmaceutical composition. The pharmaceutical composition may further comprise a pharmaceutical carrier, such as any biocompatible polymer. The pharmaceutical composition may comprise one or more other components useful for the treatment of a wound, such as but no limited to an antibiotic, and antimicrobial, an anti-inflammatory. A cosmetic composition may comprise one or other components useful for the treatment of a wound and for cosmetic application, such as none or any one or more additional component of the pharmaceutical composition, as well as a pigment or dye to mask or match with the subject's skin coloration. In some embodiments, the pharmaceutical composition or cosmetic composition may comprise a sunscreen or other UV light blocking or absorbing agent.

Thus, in some embodiments, the pharmaceutical compounds may be used to treat a wound, to accelerate healing of a wound, to reduce scar formation of a wound, to increase the strength of a wound, or to reduce keloid formation.

In some embodiments, the healing of the wound occurs earlier or more rapidly when the composition of the invention is used, compared to it not being used. In some embodiments, the wound is considered healed in 1, 2, 3, 4, 5, 6, 7, 8, 9 or 10 days earlier when the composition of the invention is used. In some embodiments, the wound is considered healed in 11, 12, 13, 14, 15, 16, 17, 18, 19 or 20 days earlier when the composition of the invention is used. In on embodiment, the wound area treated with the composition of the invention is reduced at least 5%, 10%, 15%, 20%, 25%, 30%, 35%, 40%, 45%, or 50% compared to a similar wound not treated with the composition. In one embodiment, the wound area is 30-40% smaller when treated with the composition of the invention than untreated. In any of the foregoing embodiments, the reduction in wound area may be observed at any time post injury, such as but not limited to 1, 2, 3, 4, 5, 6, 7, 8, 9, 10, 11, 12, 13, 14, 15, 16, 17, 18, 19, 20 or more than 20 days after injury.

In any of the foregoing embodiments, wound healing may be assessed by one or more criteria including but not limited to gross observation, histological assessment, or evaluation of wound content. In some embodiment, the extent of inflammation or vascularization are other criteria for assessing wound healing. In one embodiment, the appearance of the wound is a criterion for assessing wound healing. In one embodiment, the measure of open wound area is a criterion for assessing wound healing. In one embodiment, the measure of wound size by histologic evaluation is a criterion for assessing wound healing. In one embodiment, the measure of epithelial thickening is a criterion for assessing wound healing. In one embodiment, the measure of re-epithelialization is a criterion for assessing wound healing. In one embodiment, the measure of hair follicles is a criterion for assessing wound healing. In one embodiment, the measure of keratin-14-positive hair follicles is a criterion for assessing wound healing. In one embodiment, the measure of wound length is a criterion for assessing wound healing. In one embodiment, the measure of number of blood vessels per microscopic field is a criterion for assessing wound healing. In one embodiment, the measure of CD45 positive clusters per microscopic field is a criterion for assessing wound healing. In one embodiment, the measure of Herovici staining is a criterion for assessing wound healing. In one embodiment, the measure of wound size enlargement post injury is a criterion for assessing wound healing. In one embodiment, the measure of epidermal thickness is a criterion for assessing wound healing. For any of the foregoing criteria, assessment on any day post-injury is provided. For any of the foregoing criteria, assessment at or on 1, 2, 3, 4, 5, 6, 7, 8, 9, 10, 11, 12, 13, 14, 15, 16, 17, 18, 19, 20 or more than 20 days after injury is provided to show improvement in wound healing using a composition of the invention.

In one embodiment, the ratio of collagen III in normal skin is about 20%, and in injured skin, up to about 50%. In some embodiments, a method is provided for reducing a ratio of collagen III to collagen I in wounded skin during healing of a mammalian skin wound comprising a burn, the method comprising applying an amount of the cosmetic composition or pharmaceutical composition described herein effective to reducing a ratio of collagen III to collagen I during healing of a burn wound. In one embodiment the collagen III:collagen I ratio is reduced from up to about 50% to about 20%.

In some embodiments, a method is provided for reducing the collagen III content in wounded skin during healing of a mammalian skin wound comprising a burn, the method comprising applying an amount of the cosmetic composition or pharmaceutical composition described herein effective to reduce collagen III during healing of a burn wound. In one embodiment the collagen III content is reduced from up to 50% to about 20%.

In some embodiments, a method is provided for reducing a ratio of collagen III to collagen I in wounded skin during healing of a mammalian skin wound comprising a burn, the method comprising applying an amount of the cosmetic composition or pharmaceutical composition described herein effective to reducing a ratio of collagen III to collagen I during healing of a burn wound. In one embodiment the collagen III:collagen I ratio is reduced by about 5%, 10%, 15%, 20%, 25%, 30%, 35%, 40%, 45% or 50%, or more, compared to the ratio in a burn wound not treated with the composition of the invention.

In any of the foregoing embodiments, the ratio of collagen III to collagen I may be determined by Herovici stain quantification. In other embodiments, any other method may be used.

A method is provided for treating a mammalian skin wound comprising applying an amount of the cosmetic composition or pharmaceutical composition described herein effective to treat a mammalian skin wound.

A method is provided for accelerating healing of a mammalian skin wound comprising applying an amount of the cosmetic composition or pharmaceutical composition as described herein effective to accelerate healing of a mammalian skin wound.

A method is provided for decreasing inflammation and/or increasing in re-vascularization at a skin wound site, comprising applying an amount of the cosmetic composition or pharmaceutical composition described herein effective to decrease inflammation and/or increase re-vascularization at a skin wound site.

In embodiments of the methods, the skin wound comprises an abrasion.

In embodiments of the methods, the skin wound comprises a cut.

In embodiments of the methods, the skin wound comprises an excision.

In embodiments of the methods, the skin wound comprises multiple lacerations.

In embodiments of the methods, the skin wound comprises a burn.

In embodiments of the methods, the siRNA is directed against a DNA or RNA encoding human fidgetin-like 2. In embodiments of the methods, the siRNA has at least one 2′ sugar modification. In embodiments of the methods, the fidgetin like-2 comprises the amino acid set forth in SEQ ID NO:2. In embodiments of the methods, the siRNA comprises a sequence set forth in SEQ ID NOS:3, 4, 5, 6, 7, 8, 9, 10, 11 or 12.

In embodiments of the cosmetic composition or pharmaceutical composition, the siRNA is directed against a DNA or RNA encoding human fidgetin-like 2. In embodiments of the cosmetic composition or pharmaceutical composition, the siRNA has at least one 2′ sugar modification. In embodiments of the cosmetic composition or pharmaceutical composition, the fidgetin like-2 comprises the amino acid set forth in SEQ ID NO:2. In embodiments of the cosmetic composition or pharmaceutical composition, the siRNA comprises a sequence set forth in SEQ ID NOs: 3, 4, 5, 6, 7, 8, 9, 10, 11 or 12.

In some embodiments, a wound dressing structure is provided comprising a portion having thereon a composition comprising (i) a siRNA directed to fidgetin-like 2 (FL2) encapsulated by collagen microparticles in (ii) surfactant polymer dressing.

In some embodiments, wound dressing structure further comprises one or more adhesive portions which permit adhesive attachment to mammalian skin.

In some embodiments, the wound dressing structure further comprises an antibacterial or antiseptic compound mixed with the composition comprising (i) a siRNA directed to fidgetin-like 2 (FL2) encapsulated by collagen microparticles in (ii) surfactant polymer dressing.

In some embodiments, the wound dressing structure comprises woven fabric, plastic, PVC, polyethylene, polyurethane, or latex.

In some embodiments, the wound dressing structure comprises one or more adhesive portions, wherein the adhesive comprises an acrylate, methacrylate or epoxy diacrylate.

In some embodiments, the wound dressing structure does not have adhesive portions. In some embodiments, the wound dressing structure is affixed in place using tape or other means.

In some embodiments, the wound dressing composition comprising (i) a siRNA directed to fidgetin-Like 2 (FL2) encapsulated by collagen microparticles in (ii) surfactant polymer dressing is on, or adsorbed to, a skin-nonadhesive pad thereon.

In an embodiment, the surfactant polymer dressing comprises a poloxamer. In an embodiment, the poloxamer is a synthetic tri-block copolymers composed of a central hydrophobic chain of polyoxypropylene flanked by two hydrophilic chains of polyoxyethylene with a weight ratio of 4:2:4. In an embodiment, the poloxamer is poloxamer 188. In an embodiment, the poloxamer has an average molecular weight of 8400 Daltons.

In some embodiments, the surfactant polymer dressing further comprises 4-(1,1,3,3-tetramethylbutyl)phenyl-polyethylene glycol. In some embodiments, the 4-(1,1,3,3-tetramethylbutyl)phenyl-polyethylene glycol has a molecular weight of about 647 wherein 10 repeating polyethylene oxide groups are present. In some embodiments, an average of 9.5 polyethylene oxide groups are present. In some embodiments, the surfactant polymer dressing further comprises Triton-100.

In some embodiments, the surfactant polymer dressing is a poloxamer, a meroxapol, or a poloxamine.

In an embodiment, the fidgetin like-2 comprises the amino acid set forth in SEQ ID NO:2. In an embodiment, the siRNA is administered. In an embodiment, the shRNA is administered. In an embodiment, the siRNA directed against a DNA or RNA encoding human fidgetin-like 2 has at least one 2′ sugar modification. In an embodiment, the shRNA directed against a DNA or RNA encoding human fidgetin-like 2 has at least one 2′ sugar modification. In an embodiment, the siRNA or shRNA is directed against an mRNA encoding the human fidgetin-like 2. In an embodiment, the siRNA or shRNA is directed against an DNA encoding the human fidgetin-like 2 In an embodiment, the siRNA comprises a sequence set forth in SEQ ID NOS:3, 4, 5, 6, 7, 8, 9, 10, 11 or 12.

In an embodiment, the siRNA (small interfering RNA) as used in the methods or compositions described herein comprises a portion which is complementary to an mRNA sequence encoding a fidgetin-like 2 protein. In an embodiment, the fidgetin-like 2 protein is a human fidgetin-like 2 protein. In an embodiment, the mRNA is encoded by the DNA sequence NCBI Reference Sequence: NM_001013690.4 (SEQ ID NO:1), and the siRNA is effective to inhibit expression of fidgetin-like 2 protein. In an embodiment, the fidgetin-like 2 protein comprises consecutive amino acid residues having the sequence set forth in SEQ ID NO:2.

In an embodiment, the siRNA comprises a double-stranded portion (duplex). In an embodiment, the siRNA is 19-25 nucleotides in length. In an embodiment, the siRNA is 20-25 nucleotides in length. In an embodiment the siRNA comprises a 19-21 core RNA duplex with a one or two nucleotide 3′ overhang on, independently, either one or both strands. In an embodiment the siRNA comprises a 19-25 RNA duplex with a one or two nucleotide 3′ overhang on, independently, either one or both strands. The siRNA can be 5′ phosphorylated, or not, and may be modified with any of the known modifications in the art to improve efficacy and/or resistance to nuclease degradation. In an embodiment the siRNA can be administered such that it is transfected into one or more cells. In an embodiment, the siRNA is 5′ phosphorylated. In an embodiment, the whole length of the non-overlapping portion of the siRNA is fully complementary to a portion of a mRNA encoding a fidgetin-like 2 protein.

In an embodiment, the 5′ terminal residue of a strand of the siRNA is phosphorylated. In an embodiment the 5′ terminal residue of the antisense strand of the siRNA is phosphorylated. In one embodiment, a siRNA of the invention comprises a double-stranded RNA wherein one strand of the double-stranded RNA is 80, 85, 90, 95 or 100% complementary to a portion of an RNA transcript of a gene encoding fidgetin-like 2 protein. In an embodiment, the RNA transcript of a gene encoding fidgetin-like 2 protein is an mRNA. In an embodiment, the fidgetin-like 2 protein is a human fidgetin-like 2 protein. In an embodiment, a siRNA of the invention comprises a double-stranded RNA wherein one strand of the RNA comprises a portion having a sequence the same as a portion of 1825 consecutive nucleotides of an RNA transcript of a gene encoding fidgetin-like 2 protein. In an embodiment, the fidgetin-like 2 protein is a human fidgetin-like 2 protein. In yet another embodiment, a siRNA of the invention comprises a double-stranded RNA wherein both strands of RNA are connected by a non-nucleotide linker. Alternately, a siRNA of the invention can comprise a double-stranded RNA wherein both strands of RNA are connected by a nucleotide linker, such as a loop or stem loop structure. In an embodiment, both of the strands of RNA are not connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification. In embodiments, an siRNA of the invention comprises at least one 2′-O-methyl modification. In embodiments, an siRNA of the invention comprises at least one phosphorodithioate (PS2).

In another embodiment, an siRNA of the invention comprises at least one 2′-sugar modification, such as but not limited to 2′ azido-2′deoxycytidine ribonucleic acid, 2′-azido-2′deoxyuridine ribonucleic acid, 2′-azido-2′ deoxyadenosine ribonucleic acid, 2′-azido-2′-deoxyguanosine ribonucleic acid, 2′-fluoro-2′-deoxyadenosine ribonucleic acid, 2′-fluoro-2′-deoxycytidine ribonucleic acid, 2′-fluoro-2′-deoxyuridine ribonucleic acid, 2-fluorothymidine ribonucleic acid, 2′-O-methyladenosine ribonucleic acid, 2′-O-methylcytidine ribonucleic acid, 2′-O-methylguanosine ribonucleic acid, or 2′-O-methyluridine ribonucleic acid. Other nucleotide modifications are described in Chiu et al., 2003, RNA 9(9):1034-1048, and Peacock et al., 2011, J Org Chem 76(18):7295-7300, incorporated herein by reference.

As used herein, “at least one” means one or more.

In an embodiment, the FL2 is encoded by NCBI Reference Sequence:

| NM_001013690.4 (nucleic acid encoding Human fidgetin-like 2) | |
|---|---|
| (SEQ ID NO: 1) | |
| 1 | agtgagctat ggggacacta ctgcactgta gcctgggcaa cagagcaaga ccttgtctca |
| 61 | aaaatgtata tatattttgg gctttttttc ctaaaacggg aactacaaca gcatatttgc |
| 121 | gagctgatga gagtgaccca gcagagaggg aaatggatca gctctgttga agatgcactg |
| 181 | gacaccagaa cacgcccagc ccctcaacca gtggccagag cagcacctgg acgtctcctc |
| 241 | caccaccccg tcgccggccc acaagttgga gttgccccct gggggtcgcc aacgctgcca |
| 301 | ctacgcttgg gcacacgacg acatctcagc cctcactgcc tccaacctcc taaagcgcta |
| 361 | tgcagagaag tactctgggg tcttggattc tccctacgag cgtccggccc tgggcgggta |
| 421 | cagcgacgcc tccttcctca acggcgccaa aggggatccc gagccctggc cagggccgga |
| 481 | gccaccctac cccttggcct cactccacga aggcctccca ggaaccaaat cgggcggtgg |
| 541 | cggcggttcc ggggccctgg ggggctcccc agttttagcc gggaacctcc ctgaacccct |
| 601 | ctacgccggc aatgcgtgcg ggggcccatc ggcggcgccc gagtacgcgg ccggctacgg |
| 661 | cggggggtac ctggcgccgg gttactgcgc gcagacgggc gccgcgctgc ccccgccgcc |
| 721 | cccggccgcg ctcctgcagc ccccaccgcc tccggggtac gggccctcag cgccgctgta |
| 781 | caactatccc gcagggggct acgcagcgca gcccggctat ggcgcgctcc cgccgccccc |
| 841 | aggcccaccc ccggccccct acctgacccc gggcctgccc gcgcccacgc ccctgcccgc |
| 901 | gccggcaccg cccaccgcct atggcttccc cacggccgcg ccgggtgccg aatccgggct |
| 961 | gtcgctgaag cgcaaggccg ccgacgaggg gcccgagggc cgctaccgca agtacgcgta |
| 1021 | cgagcccgcc aaggcccccg tggctgacgg agcctcctac cccgccgcgg acaacggcga |
| 1081 | atgtcggggc aacgggttcc gggccaagcc gccaggagcc gcggaggagg cgtcgggcaa |
| 1141 | gtacggtggc ggcgtccccc tcaaggtcct gggctccccc gtctacggcc cgcaactgga |
| 1201 | gccctttgaa aagttcccgg agcgggcccc ggctcctcgt ggggggttcg ccgtgccgtc |
| 1261 | gggggagact cccaaaggcg tggaccctgg ggccctggag ctggtgacga gcaagatggt |
| 1321 | ggactgcggg cccccggtgc agtgggcgga tgtggcgggc cagggcgcgc tcaaggcggc |
| 1381 | gctggaggag gagctggtgt ggcccctgct caggccgccc gcctacccgg gcagcctgcg |
| 1441 | cccgccgcgg accgtcctgc tctttgggcc gcggggcgcg ggcaaagcgc tgctgggccg |
| 1501 | ctgcctcgcc acgcagctgg gcgccacgct gttgcgcctg cgcggcgcga ccctggctgc |
| 1561 | gcccggcgcc gccgagggcg cgcgcctcct ccaggccgcc ttcgcggccg cgcgctgccg |
| 1621 | cccaccctcc gtactcctca tcagcgagct agaggcgctg ctccccgccc gggacgacgg |
| 1681 | cgcggcggca gggggcgcgc tgcaggtgcc gctcctggcc tgcctggacg ggggctgcgg |
| 1741 | cgcgggggct gacggcgtgc tggttgtggg caccacctcg cggcccgcgg ctctggacga |
| 1801 | ggcgacccgc cggcgcttct ctctccgctt ctacgtggcg ctgcccgaca gcccggcccg |
| 1861 | cgggcagatc ctgcagcggg cgctggccca gcagggctgc gcgctcagtg agcgggaact |
| 1921 | ggcggcgctg gtgcagggca cgcagggctt ctctgggggc gagctggggc agctgtgcca |
| 1981 | gcaggcggcg gccggggcgg gcctcccggg gctgcagcgc cccctctcct acaaggacct |
| 2041 | ggaggcggcg ctggccaagg tgggccctag ggcctctgcc aaggaactgg actcgttcgt |
| 2101 | ggagtgggac aaaatgtacg gctccggaca ctgacggcgc gcgggggagg ccgcgggagc |
| 2161 | cgcagtccct ccgtccccgc cgcctccgcg tgggagggat gtcactgact aaacccggct |
| 2221 | ggcaggggct ggagtggtga atgtgggatc ggggacagga ggggtctgcc ggtggatatt |
| 2281 | ttttttttcg tgggaaggaa aatgcttctg ccaggcagat gccatatgcg ccgtgtactc |
| 2341 | aggtttttcc tatttattgt ggactggaag ctcgccatct ccgcccggca gaccgggcag |
| 2401 | atccggcatg ggctggcacc cggggcctta agaactcctg ctctcttgcc acaacgcttt |
| 2461 | tgtctcctcg ctatctgaat ggcaccctcc ttctccctca ctctctccat cccattctct |
| 2521 | gcattctctt ggttttctct cccttttgct ttgtcgctga cacccctgcc caccccatgc |
| 2581 | tggccctgtt tctctcctgc ccctccctcc ccagctctcc atccctcacc ctctgtgctt |
| 2641 | ctgtctccat ccctggctct ccagcgtccc tggccttttg gtccctgagc tttaatgcct |
| 2701 | ttccctgcct tctgttctta tttggactgc agtggccctt tgcaggagct ctggaggccc |
| 2761 | aggggctgag gaggagggtt acccctctac ccatctgaaa cctagggtct agggggatca |
| 2821 | aggaaaaaaa gtccccaaag aaggggaatt ttttgtttgt ttttgagggg agatcccaga |
| 2881 | aatgtagctt gtttcatatt ttagtcttct tatttttgta aaatgtgtag aatttgctgt |
| 2941 | ttttcttttt cttttgacaa ctcaggaaga aactgacctc agaaagaatg ttagactttg |
| 3001 | gctgctctcc tgtgtgcccc tcacacctgc cccctccccc ccactccatc caggggacca |
| 3061 | aattctccca gacactcaaa aaatgagact tacggggaag gggagaggaa gacccagagg |
| 3121 | cctcagtgaa accccagcta ttcctggtca gaagcagaat gtattcctaa gggcttcctc |
| 3181 | cccagggccg aggcctaggc atgaatgtgg ggagtgggct gtggggtttg agagaaggga |
| 3241 | ggccttattc ctctcctgct gctccccacc ccctgcccca cccaacccct ccgctgagtg |
| 3301 | ttttctgtga agggctatcc agagttagga tgcccttgcc caattccttc ctgagaccca |
| 3361 | gaaggtaggg tgggagggcc caaatgggaa ggtgacctaa gcagaaagtc tccagaaagg |
| 3421 | tcatgtcccc tggccctgcc ttggcagagg tccccagtga cttatgctag gaggattcca |
| 3481 | tctgggtaga cagtctggcc acaaaatcag ctactggacc tcagccatct ctgctggagg |
| 3541 | ctctgaggag gagtgagcat ccctcacttg tgggggctct gtgaggaaat gtgccttccc |
| 3601 | cattcccccg gagtcctagg tctggagctc cagggctggg agagggtgag ggagatgggc |
| 3661 | aggggtgttt tctctgacct tgggggctta gtctcagtcc tgcctgaact ttccactagg |
| 3721 | cttggaaccc ttccaagaac catatttctc tccttcccac caattttccc ttgatgaggc |
| 3781 | tttagcagtt tgctcccacc acccccagcc catttcacaa ctctgatctt agtccaaagc |
| 3841 | aggggacacg cccccccacc accacttttt ctctctccca tctcagcctc ctgtgcagtt |
| 3901 | ccttgcctgc ccgtgcattt cctagagtct actgcctccc ccctggctgg gagggtgtct |
| 3961 | gggggggatc tttcaggggc cctggcaccc agggcctgtg ctggcctagg agtgctgacc |
| 4021 | agaaggctgc tctgttcccc cccacccccg ttgctttctg gccccctctt tggagccagc |
| 4081 | cacccacagg gctttggtgc ctcagaagca gtgggctgcc gggtcacagc cgcaggctgc |
| 4141 | aaaagaccct cggagggagc atggagtgag gggttctctc tcaggtgtgt atgtattggg |
| 4201 | gggtgggggt gggtggaggg tgtcagggaa gttggggtgg gatcccagcc ttcccttcaa |
| 4261 | gaggcaggga gctctgggag gtggagtccc caccgctttc tctactaggc tcctcctgtt |
| 4321 | ccccaggctt ggggagcttt gcacaaggag actgccccca gcctagtggc acctacctca |
| 4381 | tgggctctgg ggcaggtagg ggaagggcca gtccagctct ggtaatgctg gggggaggca |
| 4441 | taccaaagaa tccaggggca gggagtgggg agggtgactt ccgagctggc ctctcccctt |
| 4501 | cctctaccca gactggggct gggatcctct cctcccgctg taaccatttc tacctcattt |
| 4561 | tgctgcgtgt tgtacatgga cgtatttatc tcctgtctga cgatgctctg cagttgtggt |
| 4621 | ctgtctacct cagaagagac tgtattttaa aagaaagtat tacacagtat taaagcgatg |
| 4681 | acatgtggtt tgcaaaaaaa aaaaaaaaaa a. |

In an embodiment, the FL2 protein sequence comprises:

(SEQ ID NO: 2)
MHWTPEHAQPLNQWPEQHLDVSSTTPSPAHKLELPPGGRQRCHYAWAHDD
ISALTASNLLKRYAEKYSGVLDSPYERPALGGYSDASFLNGAKGDPEPWP
GPEPPYPLASLHEGLPGTKSGGGGGSGALGGSPVLAGNLPEPLYAGNACG
GPSAAPEYAAGYGGGYLAPGYCAQTGAALPPPPPAALLQPPPPPGYGPSA
PLYNYPAGGYAAQPGYGALPPPPGPPPAPYLTPGLPAPTPLPAPAPPTAY
GFPTAAPGAESGLSLKRKAADEGPEGRYRKYAYEPAKAPVADGASYPAAD
NGECRGNGFRAKPPGAAEEASGKYGGGVPLKVLGSPVYGPQLEPFEKFPE
RAPAPRGGFAVPSGETPKGVDPGALELVTSKMVDCGPPVQWADVAGQGAL
KAALEEELVWPLLRPPAYPGSLRPPRTVLLFGPRGAGKALLGRCLATQLG
ATLLRLRGATLAAPGAAEGARLLQAAFAAARCRPPSVLLISELEALLPAR
DDGAAAGGALQVPLLACLDGGCGAGADGVLVVGTTSRPAALDEATRRRFS
LRFYVALPDSPARGQILQRALAQQGCALSERELAALVQGTQGFSGGELGQ
LCQQAAAGAGLPGLQRPLSYKDLEAALAKVGPRASAKELDSFVEWDKMYG
SGH.

In an embodiment, the FL2 is naturally occurring variant having 95% or greater identity with NCBI Reference Sequence: NM_001013690.4 (SEQ ID NO:1). In an embodiment, the FL2 is naturally occurring variant having 96% or greater identity with NCBI Reference Sequence: NM_001013690.4 (SEQ ID NO:1). In an embodiment, the FL2 is naturally occurring variant having 97% or greater identity with NCBI Reference Sequence: NM_001013690.4 (SEQ ID NO:1). In an embodiment, the FL2 is naturally occurring variant having 98% or greater identity with NCBI Reference Sequence: NM_001013690.4 (SEQ ID NO:1). In an embodiment, the FL2 is naturally occurring variant having 99% or greater identity with NCBI Reference Sequence: NM_001013690.4 (SEQ ID NO:1).

In embodiments, the siRNA comprise one of the following pairs of sense/antisense sequences:

(SEQ ID NO: 3)

| | |
|---|---|
| Sense: | UUACACAGUAUUAAAGCGAUU |

(SEQ ID NO: 4)

| | |
|---|---|
| Antisense: | 5′ UCGCUUUAAUACUGUGUAAUU; |

or
(SEQ ID NO: 5)

| | |
|---|---|
| Sense: | CAUCUGAAACCUAGGGUCUUU |

(SEQ ID NO: 6)

| | |
|---|---|
| Antisense: | 5′ AGACCCUAGGUUUCAGAUGUU; |

or
(SEQ ID NO: 7)

| | |
|---|---|
| Sense: | GUGACUUAUGCUAGGAGGAUU |

(SEQ ID NO: 8)

| | |
|---|---|
| Antisense: | 5′ UCCUCCUAGCAUAAGUCACUU; |

or
(SEQ ID NO: 9)

| | |
|---|---|
| Sense: | GGUCAGAAGCAGAAUGUAUUU |

(SEQ ID NO: 10)

| | |
|---|---|
| Antisense: | 5′ AUACAUUCUGCUUCUGACCUU; |

or
(SEQ ID NO: 11)

| | |
|---|---|
| Sense: | CAGCUCGAGCCCUUUGACA[dT][dT] |

(SEQ ID NO: 12)

| | |
|---|---|
| Antisense: | 5′ UGUCAAAGGGCUCGAGCUG [dT][dT]. |

In an embodiment, the siRNA is double-stranded and comprises SEQ ID NO:3 and 4; SEQ ID NO:5 and 6; SEQ ID NO:7 and 8; SEQ ID NO:9 and 10; or SEQ ID NO: 11 and 12.

In an embodiment, the 5′ terminal residue of a strand of the siRNA is phosphorylated. In an embodiment the 5′ terminal residue of the antisense strand of the siRNA is phosphorylated. In an embodiment, the 5′ terminal residue of a strand of the siRNA is not phosphorylated. In an embodiment the 5′ terminal residue of the antisense strand of the siRNA is not phosphorylated.

In an embodiment, the compositions comprise further active ingredients suitable for accelerating recovery from a skin wound, for example one or more antibiotics, antiseptics, vitamins, anesthetics, antihistamines, anti-inflammatory agents, moisturizers, penetration-enhancing agents and/or anti-irritants. In an embodiment, the compositions do not comprise further active ingredients suitable for accelerating recovery from a skin wound, for example one or more antibiotics, antiseptics, vitamins, anesthetics, antihistamines, anti-inflammatory agents, moisturizers, penetration-enhancing agents and/or anti-irritants.

In an embodiment of the methods and compositions described herein the subject is a mammal. In an embodiment the subject is human.

All combinations of the various elements described herein are within the scope of the invention unless otherwise indicated herein or otherwise clearly contradicted by context.

The disclosures of all publications, patents, patent application publications and books referred to in this application are hereby incorporated by reference in their entirety into the subject application to more fully describe the art to which the subject invention pertains.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

Herein it is demonstrated that the incorporation of FL2 siRNA collagen microparticles into SPD (SPD-FL2-siRNA) significantly enhances healing outcomes compared to controls (SPD without microparticles and SPD-Control-siRNA), as measured clinically and histologically in two murine wound healing models, namely full-thickness excision and full-thickness burn. Following SPD-FL2-siRNA treatment, there was a decrease in inflammation and an increase in re-vascularization at the wound site, while cell proliferation was unaffected. Wound healing occurred more rapidly and with high fidelity, resulting in properly organized collagen substructure. Taken together, these findings indicate that incorporation of FL2 siRNA into existing treatment options is a novel wound management therapy.

### Materials and Methods

Surfactant polymer dressing (SPD): A non-ionic surfactant polymer dressing (SPD; PluroGel®, Medline Industries, Inc.) was used for siRNA delivery.

siRNA: Sequences targeting mouse FL2 (Sigma-Aldrich, SASI_Mm02_00354635), or a non-targeting negative control siRNA (Sigma-Aldrich, SIC001) were used.

| 5′-3′: | |
|---|---|
| (SEQ ID NO: 11) | |
| Sense: | CAGCUCGAGCCCUUUGACA[dT][dT] |
| (SEQ ID NO: 12) | |
| Antisense: | UGUCAAAGGGCUCGAGCUG [dT][dT] |

Collagen microparticle and SPD incorporation: 10 g of Sodium bis(2-ethylhexyl) sulfosuccinate (AOT) (Sigma-Aldrich) was dissolved in 34 ml of n-hexane and 2 ml of 5% collagen-I dissolved in acetic acid was added. The resulting microemulsion was stirred for 45 min until it became clear. This solution was then evaporated to remove the hexane. The residue was washed and then suspended in nuclease free water and lyophilized. The 100 mg of lyophilized powder was then treated with 1000 μl of 25 μM siRNA solution and re-lyophilized. This material was then suspended in 1.25 mL of SPD, at 4 degrees for 2 hours, and then lyophilized. The lyophilized powder was then added with 1.25 mL of nuclease free water and 1.25 mL of SPD.

In vivo studies: Animal experiments were performed according to the guidelines published by the Institute of Laboratory Animal Resources of National Research Council and animal care for this study was approved by the Institutional Animal Care and Use Committee of the Albert Einstein College of Medicine.

Punch biopsy excision: Prior to procedures, mice were anesthetized with a ketamine-xylazine cocktail. Female Balb/c mice (6-8 weeks; National Cancer Institute, Frederick, Md.) were shaved then wounded uniformly on their dorsa using a 5 mm punch biopsy tool. To counteract wound contraction typically seen in mice, we used an established silicone splinting method to encourage the formation of granulation tissue and re-epithelialization (Dunn et al., 2013, J Vis Exp 75:e50265). Splints were checked multiple times a day during the study and replaced as needed. Wounds were treated with 10 uL of either SPD, SPD-Control-siRNA or SPD-FL2-siRNA on days 0, 2, and 4.

Burn protocol: Prior to procedures, mice were anesthetized with a ketamine-xylazine cocktail. Female Balb/c mice (6-8 weeks; National Cancer Institute, Frederick, Md.) were shaved then burned uniformly on their dorsa using a 5 mm brass probe heated to 100° C. Mice were provided buprenorphine as needed following injury. Wounds were treated with 10 μL of either SPD, SPD-Control-siRNA or SPD-FL2-siRNA on days 0, 2, and 4.

Wound measurements: Wounds were measured by investigators blinded to the treatment groups using surgical calipers. Measurements were made in four planes (N-S; WE; NE-SW; NW-SE), tabulated and compared to Day 0. Histologically, wound edges were defined by the distance between the first hair follicle encountered at each end of the wound.

Histochemistry: Wound sites were harvested, fixed overnight in HistoChoice, bisected through the center of the wound, and embedded in paraffin blocks. 7 μm sections were cut and slides were deparaffinized, washed in PBS-0.01% TritonX100, unmasked with antigen retrieval buffer Tris/EDTA pH 9.0, then blocked with 5% NGS in PBS. Sections were stained overnight with their respective antibody and visualized using a DAB kit (Vector Labs).

Microscopy: Whole wound sections were imaged with a P250 High Capacity Slide Scanner (3D Histech) using brightfield with a 20× air objective NA=0.8, and CMOS color camera (VCC-FC60FR19CL, CIS Corp.). Measurements of wound sizes were made using the CaseViewer analysis software (3D Histech). The size of wounds was determined by measuring the maximum length between wound edges. Quantification of PCNA, CD45, and PECAM1 were conducted as described elsewhere [Jeschke et al., 2000; Lab Invest 80:151-158; Suga et al., 2014; Stem Cells 32:1347-1360]. Herovici analysis was conducted following the previously described approach [Turner et al., 2013, J Tissue Eng Regen Med 7:139-148; Rawlins et al., 2006; J Burn Care Res 27:60-65].

Statistical Analysis: Data are expressed as mean±SEM. Differences between treatment groups were calculated using unpaired student's, Mann-Whitney nonparametric t-tests or two-way ANOVAs. Significance was set at p<0.05.

Commercial antibodies and stains: Commercially produced antibodies used in immunohistochemistry included PECAM-1 (1:50) (Abcam, ab28364), CD45 (1:200) (Abcam, ab10558), PCNA (1:1000) (Abcam, ab152112), Keratin 14 (1:500) (BioLegend, 906004). Alternatively, sections were stained with Hematoxylin (Ricca Chemical Company, #3530-32) and Eosin (Acros Organics, #17372-87-1) or Herovici (American MasterTech, #KTHER) following the manufacturer's suggested protocol.

Quantitative PCR: Skin of SPD, SPD-Control-siRNA and SPD-FL2-siRNA treated mice was excised at set timepoints after wounding. The Next Advance Bullet Blender was used to pulverize the tissue and RNA was extracted with Trizol reagent (Invitrogen, 15596-026). Complementary DNA was synthesized the same day using the SuperScript IV First-strand synthesis system (Invitrogen, 18091050). Quantitative PCR was performed using Life Technologies Universal Master Mix II (4440040) with FIGNL2 primers (Mm.PT.58.21940655, IDT Technologies). ActB (actin) served used as the reference gene (Mm.PT.58.33540333, IDT Technologies). Resulting data were quantified using the comparative 2-ΔΔCt method. The average of control skin wounds was normalized to 1 and used for relative quantification.

### Results

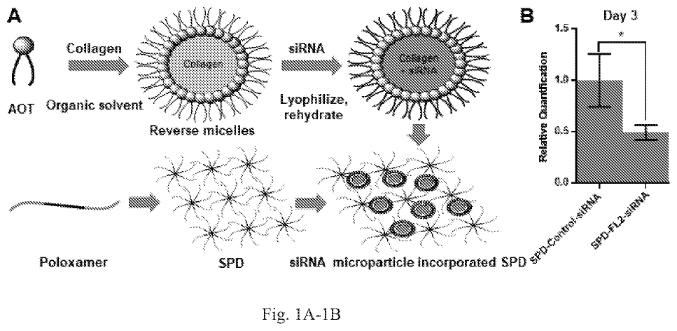

SPD-FL2-siRNA knocks down FL2 mRNA abundance. Delivery of FL2 targeting siRNA to the cells within the wound zone was achieved by direct topical application of our therapeutic surfactant polymer containing siRNA microparticles (FIG. 1A). Reduction of FL2 levels was confirmed by qPCR analysis on injured skin 3 days post initial treatment (FIG. 1B). Wounds that received FL2 siRNA had a 51% reduction in FL2 mRNA levels when compared to their control counterpart (SPD with collagen microparticles containing non-targeting siRNA). These data indicate that FL2 siRNA is effectively being delivered and is causing degradation of FL2 mRNA transcripts at the site of application.

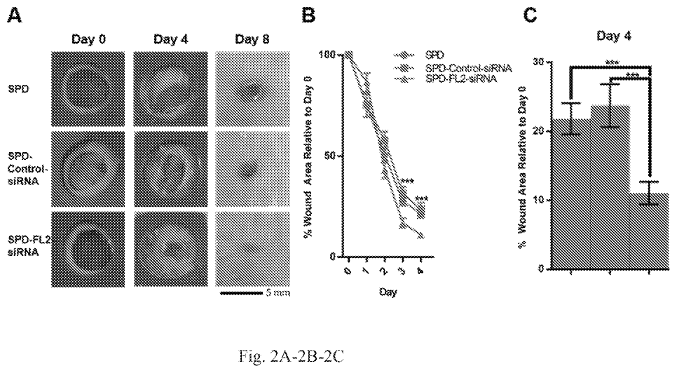

SPD-FL2-siRNA expedites excisional wound re-epithelialization. Following full-thickness excision, significant improvements in wound size were first noted in SPD-FL2-siRNA treated wounds 3 days post wounding (FIG. 2A,B). On day 4, open wound areas of SPD alone and SPD-Control-siRNA treated mice (21.8% and 23.7% open, respectively) were nearly twice the size of SPD-FL2-siRNA treated wounds (11.04% open) (n=22 wounds/group) (FIG. 2C). Treatments continued through day 6 at which point wounds became too small to measure.

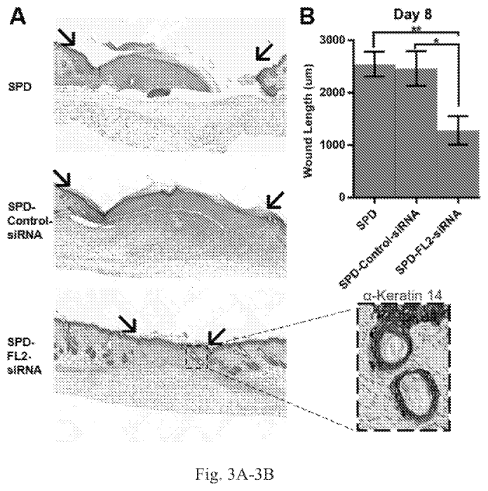

Histological analysis of wounds confirmed the significant reduction in wound size following SPD-FL2-siRNA treatment; SPD-FL2-siRNA wounds were reduced to about half the length of controls (average wound length of 2.5+/−0.2 mm for SPD, 2.4+/−0.3 mm for SPD-Control-siRNA, and 1.2+/−0.3 mm for SPD-FL2-siRNA treatments respectively) (FIG. 3A,B). Furthermore, we noted that in a few cases ( 2/7), SPD-FL2-siRNA treated wounds had hair follicles present within the wound zone, as confirmed by keratin 14 staining (FIG. 3A inset). No such structures were observed in controls. These clinical and histological assessments indicate SPD-FL2-siRNA is more effectively stimulating reepithelialization and regeneration following full-thickness excision compared to controls.

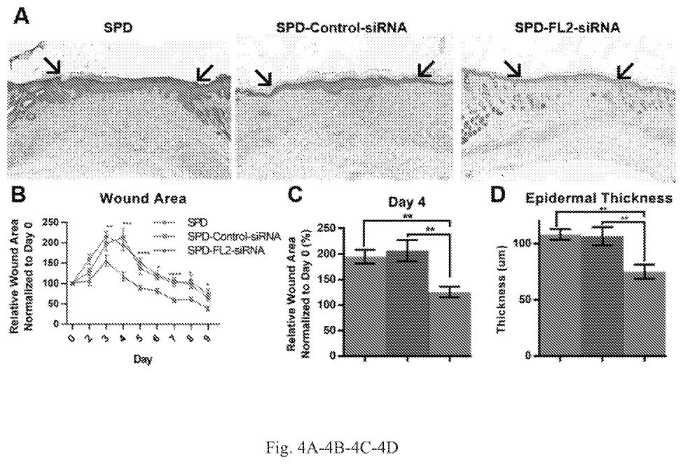

SPD-FL2-siRNA improves burn wound healing. The effect of SPD-FL2-siRNA on wound healing was further tested in a full thickness burn model. Burns were measured by two investigators blinded to the treatment groups using surgical calipers every day and tracked until closure (FIG. 4A,B). The wound sites enlarged in size for four days post injury, increasing to up to 215% the size of the original burn area in control groups. SPD-FL2-siRNA treated burns, however, did not enlarge as dramatically, reaching a maximum of 160% the size of the original burn area. The reduced burn expansion was followed by significantly improved healing from Day 4 to Day 9 when compared to SPD and SPD-Control-siRNA treated burns (FIG. 4B,C). Due to scabbing, burns became too difficult to measure using surgical calipers following Day 9. Histological analysis of wound tissue 14 days post-injury showed that SPD-FL2siRNA treated burns underwent a significant reduction in burn wound area (SPD and SPD-Control-siRNA were 35.5% and 39.3% larger, respectively, data not shown). All SPD-FL2-siRNA treated burns had closed completely at day 14 whereas 25% and 30% of SPD and SPD-Control-siRNA treated wounds, respectively, remained unhealed (data not shown). Furthermore, SPD-FL2-siRNA treated burns had a significant reduction in epidermal thickness compared to controls, more closely resembling normal skin (FIG. 4D). These clinical and histological assessments indicate that SPD-FL2-siRNA promotes re-epithelialization and regeneration of wound tissue following full-thickness burns.

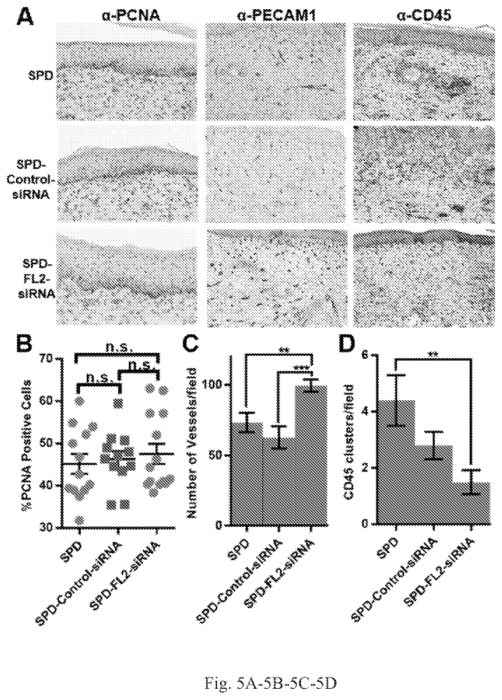

SPD-FL2-siRNA improves regeneration of the skin. Next characterized was the quality of the regenerated tissue through immunohistochemical staining. First FL2's role in cell proliferation was investigated as an increase cell proliferation could potentially account for the shortened re-epithelialization time. Proliferating cell nuclear antigen (PCNA) staining of burn wound sections revealed that there was no significant difference in the percentage of PCNA-positive cells between the 3 treatment groups (FIG. 5A,B). These data indicate that FL2 knockdown does not affect cell proliferation during healing and suggests that cell migration is the likely driver of hastened re-epithelialization compared to controls.

Managing inflammation is an essential process for effective wound healing. To assess the impact of FL2 knockdown on inflammatory cell presence at day 14, sections were stained for CD45, a marker of leukocytes and other immune cells. CD45 staining was present in all 3 treatment groups. However, we observed that SPD-FL2-siRNA treated wounds contained fewer distinct clusters of CD45 positive cells within the burn zone than SPD and SPD-Control-siRNA groups (FIG. 5A,C). After day 7, reduction of CD45-positive cells is indicative of a return to baseline conditions.

Revascularization of the burn zone was quantified by Platelet Endothelial Cell Adhesion Molecule-1 (PECAM1) staining. Significantly more vessels per field were present in SPD-FL2-siRNA treated wounds (99.5+/−4.3 vessels/field) than SPD and SPD-Control-siRNA treatments (73.5+/−6.8 and 62.7+/−7.9 vessels/field, respectively) (FIG. 5A,D). This data suggests that FL2 knockdown may enhance angiogenesis in wounded skin to provide oxygen and nutrients to the wound site for use in the rapidly regenerating wound zone.

SPD-FL2-siRNA affects collagen III:I ratios in burn scars. In the early stages of wound healing myofibroblasts deposit collagen III in the wound zone, increasing the ratio of collagen III to collagen I. Collagen III accounts for up to 50% of total collagen in the early stages of wound healing, up from levels in uninjured skin by about 20% [24]. As the scar matures, the ratio of collagen III to collagen I decreases to approximate normal levels.

To determine whether FL2 knockdown influences the ratio of collagen III to collagen I during burn healing, burns were analyzed using Herovici stains. Herovici is routinely used to quantify the ratio of collagen I (which stains red) and III (which stains blue) through multispectral imaging. Our results indicate that there are increased levels of mature collagen (reduction in collagen III to collagen I ratio) in SPD-FL2-siRNA treated burn tissues, which further supports our hypothesis that FL2 knockdown accelerates wound healing.

### ABBREVIATIONS AND ACRONYMS

ActB—Actin
ANOVA—Analysis of variance
AOT—Sodium 1,4-bis(2-ethylhexyl) sulfosuccinate
CD45—Cluster of differentiation 45
CMOS—Complementary metal-oxide-semiconductor
DAB—3,3′-diaminobenzidine
ECM—extracellular matrix
EDTA—Ethylenediaminetetraacetic acid
FIGNL2—Fidgetin-like 2
FL2—Fidgetin-like 2
MT—Microtubules
mRNA—messenger ribonucleic acid
NA—numerical aperture
NGS—Normal goat serum
N.S.—not significant
PBS—Phosphate buffered saline
PCNA—Proliferating Cell Nuclear Antigen
PECAM1—Platelet Endothelial Cell Adhesion Molecule-1
PCR—polymerase chain reaction
qPCR—quantitative polymerase reaction
SEM—Standard error of the mean
siRNA—small interfering ribonucleic acid
SPD—surfactant polymer dressing
Tris—tris(hydroxymethyl)aminomethane

What is claimed is:

1. A cosmetic composition or pharmaceutical composition comprising (i) a siRNA directed to fidgetin-like 2 (FL2) associated with, or adsorbed to, a collagen particle in (ii) surfactant polymer dressing.

2. The cosmetic composition or pharmaceutical composition of claim 1, wherein the surfactant polymer dressing comprises a poloxamer.

3. The cosmetic composition or pharmaceutical composition of claim 1, wherein the collagen particles are microparticles, a microemulsion or micelles comprising collagen.

4. The cosmetic composition or pharmaceutical composition of claim 1 wherein the collagen is collagen I.

5. The cosmetic composition or pharmaceutical composition of claim 1 further comprising one or more antibiotic, antiseptic or anti-inflammatory compound, or any combination thereof.

6. The cosmetic composition or pharmaceutical composition of claim 1, further comprising a cosmetic or pharmaceutical ingredient, component or pharmaceutical carrier.

7. The cosmetic composition or pharmaceutical composition of any one of claims 1-6, wherein the siRNA is directed against a DNA or RNA encoding human fidgetin-like 2.

8. The cosmetic composition or pharmaceutical composition of any one of claims 1-6, wherein the siRNA has at least one 2′ sugar modification.

9. The cosmetic composition or pharmaceutical composition of any one of claims 1-6, wherein the fidgetin like-2 comprises the amino acid set forth in SEQ ID NO:2.

10. The cosmetic composition or pharmaceutical composition of any one of claims 1-6, wherein the siRNA comprises a sequence set forth in SEQ ID NOS:3, 4, 5, 6, 7, 8, 9, 10, 11 or 12.

11. A method of reducing a ratio of collagen III to collagen I in wounded skin during healing of a mammalian skin wound comprising a burn, the method comprising applying an amount of the cosmetic composition or pharmaceutical composition of claim 1 effective in reducing the ratio of collagen III to collagen I during healing of a burn wound.

12. A method of treating a mammalian skin wound comprising applying an amount of the cosmetic composition or pharmaceutical composition of claim 1 effective to treat a mammalian skin wound.

13. A method of accelerating healing of a mammalian skin wound comprising applying an amount of the cosmetic composition or pharmaceutical composition of claim 1 effective to accelerate healing of a mammalian skin wound.

14. A method of decreasing inflammation and/or increasing in re-vascularization at a skin wound site, comprising applying an amount of the cosmetic composition or pharmaceutical composition of claim 1 effective to decrease inflammation and/or increase re-vascularization at a skin wound site.

15. The method of any one of claims 12-14, wherein the skin wound comprises an abrasion.

16. The method of any one of claims 12-14, wherein the skin wound comprises a cut.

17. The method of any one of claims 12-14, wherein the skin wound comprises an excision.

18. The method of any one of claims 12-14, wherein the skin wound comprises multiple lacerations.

19. The method of any one of claims 12-14, wherein the skin wound comprises a burn.

20. A wound dressing structure comprising a portion having thereon a composition comprising (i) a siRNA directed to fidgetin-like 2 (FL2) encapsulated by collagen microparticles in (ii) surfactant polymer dressing.

21. A wound dressing structure of claim 20, wherein the surfactant polymer dressing comprises a poloxamer.

22. A wound dressing structure of claim 20, wherein the collagen is collagen I.

23. A wound dressing structure of claim 20, further comprising one or more antibiotic, antiseptic or anti-inflammatory compound, or any combination thereof.

24. A wound dressing structure of claim 20, wherein the siRNA is directed against a DNA or RNA encoding human fidgetin-like 2.

25. A wound dressing structure of claim 20, wherein the siRNA has at least one 2′ sugar modification.

26. A wound dressing structure of claim 20, wherein the fidgetin like-2 comprises the amino acid set forth in SEQ ID NO:2.

27. A wound dressing structure of claim 20, wherein the siRNA comprises a sequence set forth in SEQ ID NOS:3, 4, 5, 6, 7, 8, 9, 10, 11 or 12.

28. The wound dressing structure of claim 20, further comprising one or more adhesive portions which permit adhesive attachment to mammalian skin.

29. The wound dressing structure of claim 20 which structure comprises woven fabric, plastic, PVC, polyethylene, polyurethane, or latex.

30. The wound dressing structure of claim 28, comprising one or more adhesive portions, wherein the adhesive comprises an acrylate, methacrylate or epoxy diacrylate.

31. The wound dressing structure of claim 20, wherein the composition comprising (i) a siRNA directed to fidgetin-like 2 (FL2) encapsulated by collagen microparticles in (ii) surfactant polymer dressing is on, or adsorbed to, a skin-nonadhesive pad thereon.
